(19) United States
(12) Patent Application Publication
(10) Pub. No.: US 2005/0014734 A1
(43) Pub. Date: Jan. 20, 2005

(54) Modulation of interleukin-10 by DHEA

(75) Inventor: Deh-Ming Chang, Taipei (TW)

Correspondence Address: QUINE INTELLECTUAL PROPERTY LAW GROUP, P.C., P O BOX 458, ALAMEDA, CA, 94501, US

(73) Assignee: Genovate Biotechnology Co., Ltd., Hsinchu (TW)

(21) Appl. No.: 10/623,464

(22) Filed: Jul. 18, 2003

(51) Int. Cl. A61K 31/57

(52) U.S. Cl. 514/177

(57) ABSTRACT

Methods of modulating IL-10 levels in a subject through administration of DHEA are provided herein, as are methods of prophylactically and therapeutically treating medical conditions in a subject through modulating the subject's level of IL-10 with administration of DHEA.

### FIELD OF THE INVENTION

The field of the invention concerns modulation of IL-10 in a subject by administration of DHEA.

### BACKGROUND OF THE INVENTION

Interleukin-10 (IL-10) is a multifunctional cytokine that produces a range of effects on many hemopoietic cell types and that is secreted by activated T-cells, monocytes and B-cells. It acts in regulation of growth and/or differentiation of B-cells (it is one of the most potent activators of B lymphocytes) and in the secretion of immunoglobulins. There is also indication that IL-10 can prolong B lymphocyte survival by inducing bcl-2 production, thus protecting them from programmed cell death. The IL-10 gene in humans is located on the long arm of chromosome 1 at 1q31-32 and is highly polymorphic. It was originally characterized as a cytokine-synthesis inhibitory factor because of its capacity to inhibit cytokine production by mouse Th1 cells.

IL-10 also has been shown to have potent inhibitory effects on the function of both T-cells and monocytes. IL-10 inhibits both the expression of class II MHC molecules and the production of pro-inflammatory cytokines such as IL-1, IL-6, IL-8, TNF-α, and GM-CSF by human monocytes in vitro. IL-10 also inhibits T-cell proliferation, both directly and indirectly, through diminishment of the antigen-presenting and accessory cell capacity of monocytes. Moreover, IL-10 has been shown to inhibit Ig secretion by unfractionated peripheral blood mononuclear cells (PBMC) through suppression of the accessory cell function of monocytes, although IL-10 also has direct stimulatory effects on proliferation and production of Ig by purified B-cells. See, e.g., Anaya, et al., J. Rheuma., 2002, 29(9):1874-1876; Llorente, et al., Arthritis Rheum., 1994, 37(11):1647-1655; and, Isomäki, et al., Arthritis Rheum., 1996, 39(3):386-395.

Abnormalities of IL-10 secretion have been observed in a number of serious chronic medical conditions. Such conditions include, e.g., systemic lupus erythematosus (SLE), rheumatoid arthritis (RA), multiple myeloma, lymphoid malignancy, B-cell lymphoma, Sjögren's syndrome, scleroderma, allergic diseases, HIV/AIDS and plasma cell dyscrasia. These medical conditions can be quite debilitating to persons facing them and current treatment regimens for them often meet with limited success. Hence, the search continues for alternative treatments.

One example of a medical condition involving elevated IL-10 levels is systemic lupus erythematosus (SLE or lupus). Serum levels of IL-10 have been reported to correlate with SLE disease activity. See, Llorente, et al., Arthritis Rheum., 2000, 43(8):1790-1800, and Park, et al., Clin. Exp. Rheumatol., 1998, 16(3):283-8. SLE is a prototypic systemic autoimmune disease characterized by alterations in T-cell, B-cell, and accessory cell function that facilitates polyclonal B-cell activation, autoantibody production, and an inflammatory response in different organs, e.g., the heart, lungs, skin, joints, kidneys, nervous system, lymph gland system, blood cells and/or blood vessels. See, e.g., American College of Rheumatology Ad Hoc Committee on Systemic Lupus Erythematosus Guidelines, Arthritis Rheum., 1999, 42:1785-96. Although the etiology of lupus is unknown, it is thought that hormonal influences may play a key role in disease development and progression. Several studies have noted alterations in estrogen and androgen metabolism in patients with lupus, including decreased levels of androgens (e.g., androstenedione, dehydroepiandrosterone, dehydroepiandrosterone sulfate and testosterone) in female lupus patients, especially in those with active disease. See, e.g., Lahita, et al., Arthritis Rheum., 1987(30):241-8 and Jungers et al., Arthritis Rheum., 1982(25):454-7.

As in other autoimmune disorders, autoimmunity in lupus occurs when a subject's immune system produces antibodies to various normal, or “self” cells in the body. These antibodies can lead to, e.g., inflammation of normal tissue which, in turn, can result in damage and loss of function. Additionally, antibodies can attach to antigens within the blood plasma of a subject and form immune complexes that can be deposited in normal tissue and result in inflammation and damage.

Many investigators have presented data demonstrating that T-cells and T-cell-derived cytokines play a critical role in driving B-cell differentiation and autoantibody production in lupus. See, Handwerger, et al., Springer Semin. Immunopathol., 1994, 16:153-80 and al-Janadi, et al., J. Clin. Immunol., 1993(13):58-67. In addition, increasing evidence suggests that pro-inflammatory cytokines, e.g., interleukin-1 (IL-1), and tumor necrosis factor a (TNF-α), play an important role in promoting tissue damage in lupus. See, Takemura, et al., Virchows Archiv., 1994(424):459-64.

Recently, clinical data has shown a decrease in IL-10, and concomitant patient improvement, in SLE patients through use of monoclonal antibodies specific for IL-10. Other research suggests that other medical conditions could be improved though anti-IL-10 methodologies.

Dehydroepiandrosterone (DHEA), an adrenal steroid, is secreted primarily as its sulfated metabolite, DHEA sulfate (DHEA-S). See, e.g., Parker, Endocrinol. Metab. Clin. North Am., 1991 20:401-21. DHEA is believed to have mild intrinsic androgenic properties and, in peripheral tissues, both it and DHEA-S can be converted to various other androgens, including androstenedione, testosterone, and estrogen steroids. See, Ebeling, et al., Lancet, 1994 (343):1479-81 and Labrie, et al., Ann. N.Y. Acad. Sci., 1995(774):16-28.

Prior clinical studies, one open-label and the other double-blind, placebo-controlled, suggested that DHEA administered orally as daily doses of 200 mg is well-tolerated and may have steroid-sparing effects and reduce flares in patients with mild to moderate SLE. See, van Vollenhoven et al., Arthritis Rheum., 1994(37):1305-10 and 1995(38):1826-31. Furthermore, in a subsequent multi-center phase II/III double-blind, randomized, placebo-controlled study for women with active lupus disease activity, DHEA given at 200 mg/day enabled prednisone reduction to physiologic levels in a significantly greater proportion of patients than placebo, while maintaining or improving overall SLE disease activity. See, Petri et al., Arthritis Rheum., 2002 (46):1820-9. In another double-blind study in which 381 women with mild/moderate SLE were treated with 200 mg/day DHEA or a placebo for 12 months, it was demonstrated that DHEA improved or stabilized SLE disease and its symptoms without clinical deterioration and prevented loss of bone mineral density in a significantly greater proportion of patients than placebo. See, Mease, et al., Arthritis Rheum., 2000, 43(Suppl. 1):S271 and Petri et al., Rheumatology, 2003:42(Suppl. 1):19. Recent studies by the inventors (see, Chang, et al., Arthritis Rheum., 2002, 46(11):2924-27), also showed that DHEA was well-tolerated and significantly reduced flares and improved patient global assessments in women with lupus. See, also, PCT/US00/06987, filed Mar. 16, 2000. However, the mechanisms of action of DHEA for lupus control were not well known.

A welcome addition to the art would be an effective and convenient treatment to decrease IL-10 levels in subjects. The present invention provides new methods for modulating IL-10 levels in subjects through administration of DHEA. Such methods are optionally used in treatment of medical conditions characterized by, or involving, IL-10 levels in need of modulation, e.g., elevated IL-10 levels.

### SUMMARY OF THE INVENTION

The present invention concerns the modulation of IL-10 levels in a subject through administration of DHEA and the use of such modulation to therapeutically and/or prophylactically treat one or more medical condition in the subject.

In some aspects, the current invention comprises a method of modulating IL-10 levels in a human subject through selecting a subject in need of a modulated IL-10 level, and administering an amount of DHEA to the subject in an amount effective to modulate the level of IL-10 in the subject. In some embodiments, such methods can also involve measuring the IL-10 in the subject before and/or after DHEA administration.

In other aspects, the invention involves a method of modulating IL-10 levels in a human subject by administering an amount of DHEA to the subject in an amount effective to modulate the subject's level of IL-10 and measuring the level of IL-10 in the subject before and/or after DHEA administration.

In typical aspects herein, modulation of IL-10 equates with a decrease in IL-10 levels in a subject (generally which subject either has an elevated level of IL-10 or a normal level of IL-10). However, in other embodiments, modulation of IL-10 can also equate with a stabilization of IL-10 levels in a subject undergoing an increase in IL-10 level or it can equate with a decrease in a rate of increase in IL-10 level in a subject experiencing an increasing IL-10 level.

In the methods herein, IL-10 levels are modulated and/or measured in a biological sample, e.g., a biological fluid, of a subject. Such fluid can optionally comprise one or more of, e.g., peripheral blood, serum, plasma, urine, vaginal fluid, semen, saliva, peritoneal fluid, lymphatic fluid, aqueous or vitreous humor, tears, pulmonary effusion or serosal fluid. Levels of IL-10 can optionally be indicated by a ratio of IL-10 secreting cells to IFN-γ secreting cells in a subject's serum, plasma, or peripheral blood. In yet other embodiments, IL-10 levels are modulated and/or measured in a biological sample which comprises a biological tissue of a subject. In typical embodiments herein, subjects are human (or a non-human primate), generally having a medical condition involving an increased or elevated IL-10 level and/or having a medical condition which would be ameliorated by a decreased level of IL-10. Such typical medical conditions can include, e.g., systemic lupus erythematosus (SLE), rheumatoid arthritis, multiple myeloma, lymphoid malignancy, B-cell lymphoma, Sjögren's syndrome, scleroderma, allergic diseases, HIV/AIDS or plasma cell dyscrasia.

In the various methods of the invention, administration of DHEA to a subject can be through one or more of a variety of routes. For example, DHEA administration can be achieved through, e.g., oral, intracerebral, intrathecal, intraperitoneal, intramuscular, intravenous, subcutaneous, transdermal (e.g., via skin-patch, cream, or topical ointment), mucosal (e.g., via suppository or transbuccal administration) or ocular administration. Furthermore, the amount of DHEA administered to a subject can optionally range from about 25 mg/day to about 300 mg/day, from about 50 mg/day to about 275 mg/day, from about 100 mg/day to about 250 mg/day, or from about 150 mg/day to about 200 mg/day. In other embodiments DHEA administration comprises administration of about 200 mg/day to a subject.

In some embodiments herein, the methods include administration of DHEA in conjunction with one or more other drug or treatment. For example, DHEA can be used with, e.g., a glucocorticoid, a monoclonal antibody specific for IL-10 or a fragment of IL-10, an immunosuppressant, an anti-malarial drug, an alkylating agent, a chemotherapeutic agent or various combinations thereof.

In the embodiments herein which comprise measuring IL-10 levels, such measuring can comprise determining a basal IL-10 level in a subject (i.e., prior to administration of DHEA) as well as determining the level of IL-10 in the subject after DHEA administration. Thus, measuring can comprise a time-course study of IL-10 levels in response to DHEA administration.

In yet other aspects herein, the invention concerns a method of prophylactically or therapeutically treating one or more medical conditions in a subject by modulating IL-10 levels through administering an amount of DHEA to the subject, measuring the level of IL-10 in the subject, and, administering at least a second amount of DHEA, based upon the level of IL-10 measured. Such methods can be used for treatment of, e.g., systemic lupus erythematosus (SLE), rheumatoid arthritis, multiple myeloma, lymphoid malignancy, B-cell lymphoma, Sjögren's syndrome, scleroderma, allergic diseases, HIV/AIDS or plasma cell dyscrasia.

These and other objects and features of the invention will become more fully apparent when the following detailed description is read in conjunction with the accompanying claims.

### DETAILED DESCRIPTION

The present invention includes methods of IL-10 modulation in subjects via DHEA administration.

The clinical study presented herein, examines the effects of dehydroepiandrosterone (prasterone, DHEA) on cytokine profiles (primarily IL-10) in adult women with active systemic lupus erythematosus (SLE). Thirty adult women with active SLE received 200 mg/day DHEA orally or a placebo for 24 weeks. Baseline prednisone (less than 10 mg/day) and other concomitant SLE medications were held constant. The serum levels of cytokines including IL-1, IL-2, interferon-γ, IL-4, and IL-10 were determined by ELISA. The mean change from baseline to 24 weeks of therapy was analyzed. See, below.

The two groups (i.e., DHEA and placebo) were well balanced for baseline characteristics. However, IL-10 demonstrated a greater and significant reduction from baseline in the DHEA treatment group. Thus, the clinical study resulted in significant reduction of IL-10 levels. This finding indicates one reason why DHEA significantly reduces lupus flares, as has been reported, and demonstrates, for the first time, the utility of DHEA in reducing IL-10 levels in human medical conditions.

Definitions

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, the preferred materials and methods are described herein. Accordingly, the terminology used herein is for the purpose of describing particular embodiments only, and is not intended to be limiting.

As used in this specification and the appended claims, the singular forms “a,” “an” and “the” include plural referents unless the context clearly dictates otherwise. Thus, for example, reference to “an administration” includes a plurality of administrations, reference to a “subject” includes multiple subjects, and the like.

A “subject” herein typically refers to a human (i.e., one in need of a modulated IL-10 level), but in some embodiments can also include, e.g., non-human primates (e.g., monkeys, apes, chimpanzees, etc.).

An “effective amount” of DHEA is an amount sufficient to modulate (typically decrease, or stabilize, or decrease a rate of increase of) a level of IL-10 in a subject. Levels of IL-10 can be monitored, e.g., by measuring amounts of IL-10 present in a biological sample (typically a biological fluid or biological tissue) from such subject. For example, IL-10 levels are optionally measured (and, thus, the effectiveness of an amount of DHEA is determined) in one or more of: peripheral blood, serum, plasma, urine, vaginal fluid, semen, saliva, peritoneal fluid, lymphatic fluid, aqueous or vitreous humor, tears, pulmonary effusion or serosal fluid. An effective amount of DHEA is also optionally measured by determination of the level of IL-10-secreting cells in relation to the level of IFN-γ-secreting cells in the subject's serum, plasma, or peripheral blood

The term “therapeutic treatment” refers to therapy which ameliorates a symptom of an existing medical condition (by affecting the symptom and/or by affecting a cause of the condition) or which prevents or reduces the progression of the condition. Correspondingly, as used herein, the term “prophylactic treatment” refers to therapy which reduces the risk that a medical condition will develop.

A “therapeutically effective” amount, e.g., of DHEA, is an amount sufficient to provide therapeutic treatment (as defined above) for a medical condition. A “prophylactically effective” amount, e.g., of DHEA, is an amount sufficient to provide prophylactic treatment as defined above for a medical condition.

The term “DHEA” or “dehydroepiandrosterone” includes pharmaceutically active acid, salt, and ester forms of DHEA, such as DHEA sulfate.

A “normal” level of IL-10 in a subject is one that represents an average measurement, however determined, in population of similar subjects corrected for age, gender, ethnicity, medical condition, etc. Such level is typically a range of IL-10 levels. An “elevated level” of IL-10 (e.g., as in SLE) is one that is, thus, higher than the normal level.

A “stabilized” level of IL-10 for a person is one that is substantially constant within a normal degree of fluctuation for that person's age, gender, etc. A person's stabilized level is preferably within the normal level or range of IL-10 for that person's age, gender, etc.

An “ELISA” is an enzyme-linked immunosorbent assay. In typical embodiments herein, an antigen (e.g., IL-10 present in a patient's serum and which is optionally linked or adsorbed onto a surface) is detected through specific interaction with a labeled antibody (or antibodies) specific for the antigen. Those of skill in the art will be familiar with various variations of ELISA which are optionally utilized in embodiments herein.

Interleukin-10 and Interleukin-10 Measurement

Normal levels of IL-10, i.e., levels in subjects not having a medical condition comprising IL-10 level abnormalities (e.g., SLE, etc.) can represent a range of IL-10 levels (both within an individual subject and within members of a group of subjects, e.g., a group comprising subjects of a similar age, gender, ethnicity, etc.). Those of skill in the art will be aware of numerous means to determine such normal, e.g., non-elevated, levels. Additionally, it will be apparent that different assays and/or different constituent subjects within a group will produce different ranges of normal levels for IL-10. Detection kits for IL-10 determination are available from, e.g., R & D Systems, Inc. (Minneapolis, Minn.), Endogen (Pierce Biotechnology, Inc., Rockford, Ill.), and Biosource International (Camarillo, Calif.) among others. For example, R & D Systems' Quantikine® HS Human IL-10 assay has found a sample serum/plasma range value of between “non-detectable” and 5.16 pg/mL (n=40, mean of detectable samples=2.00). See, kit package insert. Additionally, several research studies have determined normal IL-10 levels for groups. Fayad et al., found that serum IL-10 levels of 37 volunteers ranged from non-detectable (i.e., less than 3 pg/ml in 36 subjects) to 13.68 pg/ml. See, Fayad et al., Blood, 2001, 97(1):256-263. Additionally, Cortes et al., found IL-10 levels of between less than 5 pg/ml to 19.21 pg/ml for healthy subjects (n=50, median=less than 5 pg/ml). See, Cortes et al., Blood, 1995, 85(9):2516-2520. Perrier et al., noted circulating IL-10 levels of 9.7±6.8 pg/ml in a normal control group (n=32). See, Perrier, et al., J. Rheumatol., 2000, 27:935-9. Lacki, et al., found non-detectable levels of IL-10 (i.e., less than 5 pg/ml) in their control group of 30 healthy subjects, see, Lacki, et al., Clinical Rheumatology, 1997, 16(3):275-278, while Park, et al., found a normal range of 3.5±0.6 pg/ml of IL-10 in their control group of subjects. See, Park, et al., Clin. Exp. Rheumatol., 1998, 16(3):283-8. Those of skill in the art will be easily able to locate similar determinations and will be able to determine a normal range or level of IL-10 for subjects herein. See, also, Llorente et al., Eur. Cytokine Netw., 1993, 4:421-30 and Llorente, et al., Arthritis. Rheum., 1994, 37:1647-55.

IL-10 has been implicated in a number of human rheumatic autoimmune diseases, such as rheumatoid arthritis, Sjögren's syndrome, and SLE, that are characterized by a prominent B lymphocyte hyperactivity which results in an increased production of immunoglobulins and the emergence of auto-antibodies. Such hyperactivity seems to be involved in the development of clinical manifestations of such conditions, either by deposition of immune complexes or by recognition of self antigens. See, e.g., Llorente, supra.

IL-10 is spontaneously produced by synovial membrane cells and serum derived from patients with rheumatoid arthritis. It has been shown that activated T-cell clones from rheumatoid synovial membrane produce high levels of IL-10 when compared with T-cell clones derived from the peripheral blood. Production of IL-10 by PBMC has been shown to be higher in patients with rheumatoid arthritis, SLE, and Sjögren's syndrome than in healthy control subjects. In Sjögren's patients, IL-10 has been found to be released by T-cells from salivary glands and by PBMC. IL-10 mRNA expression is elevated in the salivary glands of Sjögren's patients. IL-10 may also play a role in the emergence of B lymphomas, particularly in the context of abnormal Epstein-Barr replication.

IL-10 is also involved in the progression of HIV infection. Studies have indicated that HIV infection stimulates IL-10 secretion (e.g., by peripheral blood mononuclear cells). This increase in IL-10 secretion, and the corresponding immunosuppressive properties of IL-10 appear to be important in HIV infection. See, e.g., Badou et al., J. Virology, 2000, 74(22):10551-10562 and Nunnari et al., Ann. Intern. Med. 2003, 139(1):26-30.

It will be appreciated that IL-10 levels can be measured and determined from a number of different cell types and/or biological samples, depending, e.g., upon the specific medical condition being examined, ease of collection of the sample, etc. IL-10 levels optionally can be determined from biological samples, e.g., fluids, such as peripheral blood, serum, plasma, urine, vaginal fluid, semen, saliva, peritoneal fluid, lymphatic fluid, aqueous or vitreous humor, tears, pulmonary effusion or serosal fluid. Alternatively, or additionally, IL-10 level are optionally determined from samples of biological tissue. Determination of IL-10 in samples is optionally through any of a number of methods which will be known to those skilled in the art.

These methods of determination may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunohistochemistry, affinity chromatography, immunoelectrophoresis, radioimmunoassay (RIA), enzyme-linked immunosorbent assays (ELISAs), immunofluorescent assays, Western blotting, and the like.

A variation of electrophoretic polypeptide separation utilizes a Western blot (immunoblot) analysis to detect and quantify the presence of IL-10 in a sample. This technique generally comprises separating samples by gel electrophoresis on the basis of molecular weight, transferring the separated polypeptides to a suitable solid support (such as a nitrocellulose filter, a nylon filter, or derivatized nylon filter), and incubating the sample with antibodies that specifically bind IL-10. Antibodies that specifically bind to IL-10 may be directly labeled, or, alternatively, may be detected subsequently using labeled antibodies (e.g., labeled sheep anti-mouse antibodies) that specifically bind to a domain of the primary antibody.

In a preferred embodiment, the IL-10 polypeptide(s) are detected and/or quantified in the biological sample using any of a number of well-known immunoassays (see, e.g., U.S. Pat. Nos. 4,366,241; 4,376,110; 4,517,288; and 4,837,168). For a general review of immunoassays, see also Methods in Cell Biology Volume 37: Antibodies in Cell Biology, Asai, ed. Academic Press, Inc. New York (1993); and Basic and Clinical Immunology 7th Edition, Stites & Terr, eds. (1991). For example, as illustrated in the Examples herein, IL-10 levels can be measured in biological samples through ELISA. Additionally, the “level” of IL-10 can be determined through comparison of the quantity of IL-10 secreting cells in relation to the quantity of IFN-γ cells in a subject's plasma, serum, etc. See, below.

Conventional immunoassays often utilize a “capture agent” to specifically bind to and often immobilize the analyte (in this case a IL-10 polypeptide). In preferred embodiments, the capture agent is an antibody.

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain (VL)” and “variable heavy chain (VH)” refer to these light and heavy chains respectively.

Antibodies exist as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)2 dimer into a Fab′ monomer. The Fab′ monomer is essentially a Fab with part of the hinge region (see, Fundamental Immunology, W. E. Paul, ed., Raven Press, N.Y. (1999), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein, also includes antibody fragments either produced by the modification of whole antibodies or synthesized de novo using recombinant DNA methodologies. Preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked VH-VL heterodimer which may be expressed from a nucleic acid including VH- and VL-encoding sequences either joined directly or joined by a peptide-encoding linker. See, Huston, et al. (1988) Proc. Nat. Acad. Sci. USA, 85:5879-5883. While the VH and VL are connected to each as a single polypeptide chain, the VH and VL domains associate non-covalently. The scFv antibodies and a number of other structures converting the naturally aggregated, but chemically separated light, and heavy polypeptide chains from an antibody V region into a molecule that folds into a three dimensional structure substantially similar to the structure of an antigen-binding site are known to those of skill in the art (see e.g., U.S. Pat. Nos. 5,091,513, 5,132,405, and 4,956,778). Antibodies useful in these immunoassays include polyclonal and monoclonal antibodies.

As those of skill in the art readily appreciate, numerous well-known protocols exist to guide design and production of antibodies. Antibodies also can be prepared by any of a number of commercial services (e.g., Berkeley Antibody Laboratories, Bethyl Laboratories, Anawa, Eurogenetec, etc.).

Immunoassays also typically utilize a labeling agent to specifically bind to and label the binding complex formed by the capture agent and the target polypeptide (i.e., IL-10). The labeling agent may itself be one of the moieties making up the antibody/target polypeptide complex. Thus, the labeling agent may be a labeled polypeptide or a labeled antibody that specifically recognizes the already bound IL-10. Alternatively, the labeling agent may be a third moiety, such as another antibody, that specifically binds to the capture agent/IL-10 complex. Other polypeptides capable of specifically binding immunoglobulin constant regions, such as polypeptide A or polypeptide G may also be used as the label agent.

Preferred immunoassays for detecting IL-10 are either competitive or noncompetitive. Noncompetitive immunoassays are assays in which the amount of captured IL-10 is directly measured. In competitive assays, the amount of IL-10 in a sample is measured indirectly by measuring the amount of an added (exogenous) polypeptide displaced (or competed away) from a capture agent by the IL-10 present in the sample. In one competitive assay, a known amount of labeled IL-10 is added to the sample, which is then contacted with a capture agent. The amount of labeled IL-10 bound to the antibody is inversely proportional to the concentration of IL-10 present in the sample.

The assays of this invention are scored (as positive or negative or quantity of IL-10) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of IL-10 concentration.

It will be appreciated that monitoring of IL-10 levels can also be accomplished through other means well known to those skilled in the art which are not listed herein. For example PCR (polymerase chain reaction) techniques can be used to detect mRNA or DNA expression of IL-10. Additionally, flow cytometry can be used to detect intracellular IL-10. The above illustrative use of ELISA, etc. should not be taken as limiting.

In preferred embodiments herein, IL-10 levels within a subject are measured prior to DHEA administration, thus, determining an IL-10 “baseline” or “basal measurement.” Such baseline can be determined through a single IL-10 measurement, or optionally, can be determined through a number of measurements which can then be averaged to produce a baseline IL-10 level or can be plotted, etc. to produce a baseline IL-10 range. In other embodiments, IL-10 levels are measured after DHEA administration. Such post-administration measurement(s) can occur at varying lengths of time after DHEA administration depending upon, e.g., DHEA dosage given, etc. In some embodiments, the IL-10 level is determined over a course of treatment with DHEA. Thus, multiple IL-10 determinations can be made as a progression of DHEA administrations are made, e.g., over a course of days, weeks, months, years, etc. In such embodiments, the DHEA regimen can be changed in response to any change in IL-10 levels and/or medical condition symptoms (i.e., which, thus, indicate a change in IL-10 levels). See, below.

Selection of Subjects in Need of IL-10 Modulation

In the various embodiments herein, subjects in need of IL-10 modulation can be chosen through a number of means. For example, measurements of IL-10 can indicate an elevated IL-10 level in the subject. Such measurements can optionally be of IL-10 itself (see, e.g., ELISA tests above) or can be of the ratio between IL-10-secreting cells and IFN-γ-secreting cells. As described previously such IL-10 measurements can be made from one or more biological sample from a subject, e.g., peripheral blood, serum, plasma, urine, vaginal fluid, semen, saliva, peritoneal fluid, lymphatic fluid, aqueous or vitreous humor, tears, pulmonary effusion or serosal fluid samples and/or biological tissue samples.

In addition to measurement of a subject's IL-10 level, selection of those in need of IL-10 modulation can also be made based upon presence and/or severity of clinical symptoms of various medical conditions involving elevated IL-10, e.g., SLE, rheumatoid arthritis, multiple myeloma, lymphoid malignancy, B-cell lymphoma, Sjögren's syndrome, scleroderma, allergic diseases, HIV/AIDS or plasma cell dyscrasia, etc.

While many of the embodiments herein are drawn to methods of IL-10 modulation concomitant with a treatment of SLE, it should be appreciated that numerous other medical conditions comprising abnormal IL-10 levels, e.g., Sjögren's syndrome, etc., are also applicable to treatment through modulation of IL-10 levels with DHEA.

As an illustration, selection of a subject through diagnosis/characterization of SLE can be based upon, e.g., gender and age. In general, women are much more susceptible to lupus than are men. Over 90% of lupus patients are females aged 13-40 years. The determination or diagnosis of lupus can be based upon laboratory tests such as the LE Cell Test, the Anti-Nuclear Antibody Test, and the test for Anti-DNA-Antibodies. Often, however, lupus is determined through clinical manifestations such as arthritis, skin changes (e.g., photosensitive induced “butterfly” rash across the face and/or discolored scaly patches on the body), hematological abnormalities (e.g., anemia, leukopenia, or thrombocytopenia), kidney impairment, heart or lung disease, and neuropsychiatric changes. Several subcategories of lupus erythematosus exist, namely, discoid lupus, subacute cutaneous lupus, drug-induced lupus and systemic lupus or SLE.

Those of skill in the art will be familiar with diagnosis and characterization of symptoms involved in medical conditions involving elevated IL-10 levels which can be used to select those subjects in need of IL-10 modification.

Adminstration and Formulation of DHEA

DHEA (dehydroepiandrosterone) is the principal steroid secreted by the fetal adrenal gland, with concentrations significantly higher than other circulating steroids. Its role in fetal physiology is poorly understood, but it is thought to serve as a precursor for other steroids, leading to androgen and estrogenic steroids.

Formulations

In order to modulate IL-10 levels, the treatment methods of the invention can employ any DHEA-like agent, typically in combination with a physiologically acceptable excipient or stabilizer that is non-toxic to recipients at the dosages employed. DHEA can be isolated in at least 6 different polymorphic forms, as described in detail in PCT Application No. PCT/US/00/06987 (International Publication No. WO 00/54763). DHEA was previously known, via analytical techniques such as x-ray diffraction, infrared (IR) spectroscopy, and differential scanning calorimetry (DSC), to occur in several different hydrate and anhydrate crystal forms. The anhydrate forms include forms I, II, III, IV, V and VI, although forms IV and V have been observed only transiently by DSC. The hydrates (solvates) include forms S1 (¼ hydrate), S2 (monohydrate), S3 (monohydrate), and S4 (½ methanolate). PCT Application No. PCT/US/00/06987 describes an additional form, form VI, which is detectable only by solid state NMR. DHEA formulations of the invention can contain any of the DHEA polymorphs, alone or in combination.

In preferred embodiments of the present invention, the DHEA employed has defined bioavailabilities and pharmacokinetic properties, which can be achieved by using preparations containing polymorphs that provide the desired properties. DHEA form I is highly stable and provides pharmaceutical compositions that retain a constant level of DHEA activity over long periods of time (e.g., more than 1 or 2 years). Accordingly, formulations enriched in form I have long shelf-lives, which is highly desirable in a pharmaceutical product. DHEA form I compositions also exhibit good uptake by the GI tract upon oral administration, and show good therapeutic activity. DHEA form II-containing preparations exhibit good uptake by the gastrointestinal tract upon oral administration, a rapid rate of absorption (greater than the form I polymorph) and good therapeutic activity, and are also stable under typical ambient conditions. Compositions thus enriched in form I or form II provide more predictable pharmacokinetic profiles than are provided by commercially available compositions having random polymorphic compositions.

Additionally, DHEA preparations useful in the treatment methods can contain mixtures of the form I and II polymorphs or other polymorphs. Generally, the combined form I and II polymorphs account for at least about 85%, preferably at least about 90%, more preferably at least about 95%, and most preferably at least about 99% of the DHEA in such preparations. Preparations enriched in form I and/or form II generally provide more predictable pharmacokinetic profiles than are provided by formulations having random polymorphic compositions. It will be appreciated, however, that the current invention is not limited by the specific DHEA formulations or DHEA forms used to modulate IL-10 levels.

Such formulations, including DHEA and precursors such as DHEA acetate, are commercially available from various sources (e.g., Sigma Chemical Co., St. Louis, Mo.; Aldrich Chemical Company, Inc.; Diosynth, Inc.; Pfaltz & Bauer, Inc.; Schering AG). DHEA formulations enriched for selected polymorphs can be prepared by crystallization of commercial DHEA in selected solvents under appropriate cooling or evaporation conditions. See, e.g., PCT/US00/06987, DHEA Composition and Method, filed Mar. 16, 2000.

DHEA formulations employed in the invention provide an effective amount of DHEA upon administration to an individual. The proportion of pharmaceutically active DHEA to excipient and/or other substances may vary from about 0.5 to about 100 wt. % (weight percent). For oral use, the pharmaceutical formulation will generally contain from about 5 to about 100% by weight of DHEA. For other uses, the formulation will generally have from about 0.5 to about 50 wt. % of DHEA.

Formulations of DHEA can include a physiologically acceptable excipient, or stabilizer. Excipients known in the art include, for example, vegetable and animal oils and fats. Stabilizing agents, wetting and emulsifying agents, salts for varying the osmotic pressure, buffers for securing an adequate pH value, and/or skin penetration enhancers can be used as auxiliary (i.e., excipient) agents in the DHEA formulations. Methods for preparing various conventional dosage forms are known or will be apparent to those skilled in the art; for example, see, Remington's Pharmaceutical Sciences (19th Ed., Williams & Wilkins, 1995).

Examples of common excipients include buffers (such as phosphate buffer, citrate buffer, and buffers made from other organic acids), antioxidants (e.g., ascorbic acid), low-molecular weight (less than about 10 residues) polypeptides, proteins (such as serum albumin, gelatin, and an immunoglobulin), hydrophilic polymers (such as polyvinylpyrrolidone), amino acids (such as glycine, glutamine, asparagine, arginine, and lysine), monosaccharides, disaccharides, and other carbohydrates (including glucose, mannose, and dextrins), chelating agents (e.g., ethylenediaminetetraacetic acid [EDTA]), sugar alcohols (such as mannitol and sorbitol), salt-forming counter ions (e.g., sodium), and/or anionic surfactants (such as Tween™, Pluronics™, and PEG).

It will be appreciated that particular excipients and formulations used can vary depending upon, e.g., the specific route of administration, other drugs given, dosage used, etc. For example, in intravenous, intramuscular or subcutaneous administration, DHEA can be incorporated into a pharmaceutically acceptable and injectable excipient. Typically, the excipient is one such as sterile water, aqueous saline solution, aqueous buffered saline solution, aqueous dextrose solution, aqueous glycerol solution, ethanol, or combinations thereof. The preparation of such solutions ensuring sterility, proper pH, isotonicity, and stability is achieved according to protocols established in the art. Generally, an excipient is selected to minimize allergic and other undesirable effects, and to suit the particular route of administration, e.g., subcutaneous, intramuscular, etc.

DHEA formulations for oral administration can be incorporated into a food or drink, or formulated into a chewable or swallowable tablet or capsule, thus, allowing rapid uptake in the bloodstream and distribution to various compartments of the body. Typically for oral administration, excipients can include pharmaceutical grades of lactose, mannitol, starch, methyl cellulose, magnesium stearate, sodium saccharine, talcum, cellulose, glucose, gelatin, sucrose, magnesium carbonate, and the like. When the composition is employed in the form of solid preparations for oral administration, the preparations may be tablets, granules, powders, capsules, etc.

In some embodiments, the invention utilizes sustained-release pharmaceutical formulations. An exemplary sustained-release formulation has a semipermeable matrix of a solid hydrophobic polymer to which the active agents of the invention are attached or in which the active agents are encapsulated. Examples of suitable polymers include a polyester, a hydrogel, a polylactide, a copolymer of L-glutamic acid and T-ethyl-L-glutamase, non-degradable ethylene-vinylacetate, a degradable lactic acid-glycolic acid copolymer, and poly-D-(−)-3-hydroxybutyric acid. Such matrices are in the form of shaped articles, such as films, or microcapsules.

In the various methods herein, DHEA may also be administered to a subject transdermally. For transdermal administration, DHEA may be conveniently incorporated into a lipophilic carrier and formulated as a topical cream or ointment or in an adhesive patch. Methods for preparing various conventional dosage forms are known or will be apparent to those skilled in the art; for example, see, Remington's Pharmaceutical Sciences (19th Ed., Williams & Wilkins, 1995). Thus, a sustained-release formulation can include liposomally entrapped active agents. Liposomes are small vesicles composed of various types of lipids, phospholipids, and/or surfactants. These components are typically arranged in a bilayer formation, similar to the lipid arrangement of biological membranes. Liposomes containing active agents are prepared by known methods, such as, for example, those described in Epstein, et al. (1985) PNAS USA 82:3688-92, and Hwang, et al., (1980) PNAS USA, 77:4030-34. Ordinarily the liposomes in such preparations are of the small (about 200-800 Angstroms) unilamellar type in which the lipid content is greater than about 30 mol. percent cholesterol, the specific percentage being adjusted to provide the optimal therapy. Useful liposomes can be generated by the reverse-phase evaporation method, using a lipid formulation including, for example, phosphatidylcholine, cholesterol, and PEG-derivatized phosphatidylethanolamine (PEG-PE). If desired, liposomes are extruded through filters of defined pore size to yield liposomes of a particular diameter.

In yet other embodiments, DHEA can be administered via mucosal administration. Mucosal administration includes such routes as buccal, endotracheal, inhalation, nasal, pharyngeal, rectal, sublingual, vaginal, etc. For administration through the mucosal membranes, the DHEA can be formulated as an emulsion, gum, lozenge, spray, tablet or the like. Nasal administration can be conducted through a powder or spray formulation. For rectal and vaginal administration the DHEA can be formulated as a cream, douche, enema or suppository, etc.

In some embodiments, DHEA is administered through ocular administration by incorporating the DHEA into a solution or suspension adapted for ocular application, e.g., drops or sprays.

Pharmaceutical formulations utilized herein can also include an active agent adsorbed onto a membrane, such as a silastic membrane, which can be implanted, as described in International Publication No. WO 91/04014.

Pharmaceutical formulations utilized by the invention can be stored in any standard form, including, e.g., an aqueous solution or a lyophilized cake. Such formulations are typically sterile when administered to subjects. Sterilization of an aqueous solution is readily accomplished by filtration through a sterile filtration membrane. If the formulation is stored in lyophilized form, the formulation can be filtered before or after lyophilization and reconstitution.

Administration

As described herein, the present invention concerns methods of modulating or changing IL-10 levels in a subject through administration of DHEA. Typically, the DHEA is given either alone or in combination (e.g., co-administration) with other treatments or medications to alter IL-10 levels and, thus, therapeutically and/or prophylactically treat one or more of a number of medical conditions. Alternatively or additionally, DHEA is given in combination with other drugs or treatments which do not alter IL-10 levels, but which are otherwise effective in the treatment of the underlying medical condition, e.g., SLE. See, below.

In the methods herein, DHEA can be administered in any of the commonly accepted manners for administration of pharmaceutical compositions. Those of skill in the art will be quite familiar with such routes and delivery protocols. For example, routes of administration for DHEA can include, but are not limited to: oral, intracerebral, intrathecal, intraperitoneal, intramuscular, intravenous, subcutaneous, transdermal, mucosal (e.g., via suppository or transbuccal administration) or ocular administration, etc. Thus, depending upon the route of administration, DHEA can be provided in various dosage forms, such as, for example, tablets, capsules, powders, controlled-release formulations, suspensions, emulsions, suppositories, creams, ointments, lotions, or aerosols. Preferred embodiments utilize solid dosage forms suitable for simple administration of precise dosages.

The subject formulations are preferably, though not necessarily, administered daily. Generally, the total daily dosage will be at least about 25 mg, at least about 50 mg, at least about 75 mg, generally at least about 100 mg, preferably at least about 150 mg, and more preferably at least about 200 mg. Alternatively, the total daily dosage will be at least 250 mg or at least 275 mg or at least 300 mg. Generally, daily oral DHEA doses do not exceed about 250 to about 300 mg, although higher doses may be used in particular circumstances. Capsules or tablets for oral delivery can conveniently contain up to a full daily oral dose, e.g., 200 mg or more. When administered by other than an oral route, the DHEA may be delivered over an extended period, e.g., 3-10 days, in an amount effective to produce at least an average daily dose (or an equivalent of an average daily dose) of, e.g., at least about 25 mg, at least about 50 mg, at least about 75 mg, generally at least about 100 mg, preferably at least about 150 mg, and more preferably at least about 200 mg, at least about 225 mg, at least about 250 mg, at least about 275 mg, or at least about 300 mg.

Co-Administration of DHEA and Other Drugs

If desired, administration of DHEA in the methods herein can be performed in conjunction with administration of one or more other drug. The DHEA can be administered in the same formulation as such other drug, or can be administered separately (e.g., at separate times, in different formulations, according to different schedules, in response to different criteria, etc.).

DHEA is optionally co-administered with a number of different drugs. For example, in some embodiments, DHEA is co-administered with drugs that also act to modulate IL-10 levels in a subject. Thus, monoclonal antibodies specific for a specific epitope of IL-10 (see, e.g., Llorente, et al., Arthritis Rheum., 2000, 43(8):1790-1800), or even a combination of antibodies specific for different epitopes of IL-10, can be administered along with DHEA in order to modulate IL-10 levels.

Alternatively, DHEA is optionally co-administered with drugs that do not specifically act to modulate IL-10 levels in a subject, but which are effective in treating an underlying medical condition in a subject, i.e., typically the same medical condition for which the modulation of IL-10 through DHEA is sought. For example, in the treatment of SLE, DHEA is optionally administered along with any of a number of common treatments, such as aspirin, salisylates, ibuprofen, naproxen, sulindac (e.g., Clinoril™), oxaprozin and tolmetin for fever, joint pain and inflammation. Antimalarial drugs such as hydroxychloroquine, chloroquine and quinacrine are often indicated for the various skin abnormalities involved, as are retinoids. Corticosteroids, typically prednisone, are often administered for organ inflammation, etc. Some androgenic compounds, e.g., danazol (e.g., Danocrine™) have also been used in controlling immune thrombocytopenia and severe hemolytic anemia. Additionally, immunosuppressive drugs such as azathioprine, cyclosporin A, mycophenolate mofetil, alkylating agents and methotrexate (all optionally in combination with corticosteroids) can be used in treatment of SLE.

Furthermore, DHEA can also be administered along with drugs that are effective for secondary conditions arising from an underlying medical condition or even arising from the treatment for the underlying medical condition. For example, in some embodiments, DHEA can be administered along with calcitonin to help treat bone density loss arising from treatment of SLE (e.g., arising from use of prednisone, methotrexate, immunosuppressants, anti-inflammatories, etc.). See, Treatment of Subnormal Bone Mineral Density, U.S. Ser. No. 09/710,729, filed Nov. 10, 2000.

Time-Course and Adjustment of Dosage

The range of DHEA dosages and dose rates effective for achieving the desired modulation of IL-10 levels in a subject (and, thus, optionally an effective treatment of a medical condition) may be determined in accordance with standard industry practices. These ranges can be expected to differ depending upon whether the desired response is the prophylactic, therapeutic or curative treatment of the medical condition (e.g., SLE, Sjögren's syndrome, scleroderma, allergic diseases, HIV/ADS, etc.), the type or severity of symptoms, other medications being administered, the age, gender, medical history and other individual parameters of the subject being treated, etc. In one embodiment, DHEA dosage can be determined based upon changes produced in IL-10 levels, as measured, e.g., in changes to the ratio of IL-10 secreting cells to IFN-γ secreting cells. To determine IL-10 levels in a subject, typical embodiments herein measure, e.g., via ELISA, the IL-10 levels in any one or more of a biological tissue, peripheral blood, serum, plasma, urine, vaginal fluid, semen, saliva, peritoneal fluid, lymphatic fluid, aqueous or vitreous humor, tears, pulmonary effusion or serosal fluid. See, above.

In typical embodiments, approximately 200 mg/day DHEA are administered in the methods herein. Again, however, such dosage can be modified (both in amount and regimen) based upon, e.g., the above criteria. Those skilled in the art will be familiar with individual tailoring of treatment regimes to effectuate the desired outcome in various subjects. Thus, in many embodiments, while a dosage of 200 mg/day DHEA is used as either a starting point or a target level, such dosage is optionally adjusted based on specific factors of the subject receiving treatment. For example, the DHEA dosage can be increased if the desired IL-10 level is not reached, or if the desired IL-10 level is achieved, the DHEA dosage can be tapered down to find the lowest level that will achieve stability at the desired IL-10 level.

The DHEA dosage can also be adjusted based upon symptoms of the underlying medical condition being treated. For example, if the IL-10 level is being decreased in subject having SLE, then SLE symptoms (e.g., time to flare) are optionally used as guidelines or indicators for DHEA dosages (amounts and time courses). Thus, in some embodiments, evaluations of the severity of SLE, e.g., as measured by time intervals between SLE flares, can be used as indirect measurement of IL-10 modulation through DHEA administration. Such indirect measurements are typically used in conjunction with other measurements of IL-10 modulation (e.g., direct measurement of IL-10 levels in tissues or biological fluids, etc.).

Additionally, if the IL-10 is being modulated in a subject having, e.g., Sjögren's syndrome (SS), then SS symptoms can be used as guidelines for the DHEA regimen which modulates the IL-10 levels. For example, tests tracking SS symptoms, e.g., Schirmer test for tear production or Salivary Scintigraphy to measure salivary gland function can be used to monitor SS symptoms and, therefore, the modulation of IL-10 levels through administration of DHEA. Those of skill in the art will be aware of other tests/diagnostic scales capable of use to monitor symptoms in medical conditions involving elevated or abnormal IL-10 levels.

Kits and Reagents

The DHEA formulation of the invention is optionally provided to a user as an article of manufacture. The invention also provides articles of manufacture including formulations used in the methods of the invention. The invention encompasses any type of article including a formulation of the invention, but the article of manufacture is typically a container, preferably bearing a label identifying the formulation contained therein. The container can be formed from any material that does not react with the contained formulation and can have any shape or other feature that facilitates use of the formulation for the intended application.

The invention also includes kits. A kit of the invention contains one or more dosage of DHEA in an appropriate formulation in a suitable container (e.g., in terms of storage and administration route). The kit typically further comprises one or more additional reagents, e.g., excipients, and/or other accessories, reagents for collecting samples, buffers, etc.

Kits of the invention preferably include instructions for use. The instructions can be affixed to the packaging material or can be included as a package insert. While the instructions typically comprise written or printed materials they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), and the like. As used herein, the term “instructions” can include the address of an internet site that provides the instructions. Thus, the kit optionally further comprises an instruction set or user manual detailing preferred methods of using the kit components for modulation of IL-10 levels, measurement of IL-10 levels, etc.

When used according to the instructions, the kit can be used, e.g., for determining a subject's IL-10 level (and optionally tracking any change in such following treatment with DHEA), for evaluating a disease state or condition, for evaluating effects of a pharmaceutical agent (i.e., DHEA) or other treatment intervention on progression of a medical condition in a subject, etc.

### EXAMPLE I

The current use of DHEA to modulate IL-10 levels was examined in a randomized, double-blind, and placebo-controlled study conducted as a substudy within a larger multicenter study. See, Chang et al., Arthritis Rheum., 2002, 46(11):2924-27. The subjects enrolled in the study were adult women with SLE according to American College of Rheumatology (ACR) criteria (see, e.g., Tan, et al., Arthritis Rheum., 1982, 25:1271-7) and who were receiving a prednisone dose (or its corticosteroid equivalent) of 1 to 10 mg/day at the time they entered the study. Subjects selected for the study had to have active SLE, as defined by a Systemic Lupus Activity Measure (SLAM) score of ≧7, see, e.g., Liang, et al., Arthritis Rheum., 1989, 32:1107-18, and a baseline Systemic Lupus Erythematosus Disease Activity Index (SLEDAI) score ≧2. See, Bombardier, et al., Arthritis Rheum., 1992, 35:630-40.

Subjects that were under treatment with dydroxychloroquine, azathioprine, methotrexate, or cyclophosphamide or a combination thereof, had to have been on a stable regimen with no change in dose and drug combination for at least 6 weeks prior to entry into the study. Subjects who were receiving androgens, immunoglobulins, cyclosporine A, or other immunosuppressive agents (except those noted) were excluded from the study. After a 10-day screening and qualifying baseline period, patients were assigned by predetermined randomization code to receive either 200 mg/day DHEA (provided by Genelabs Biotechnology Co., LTD) or a placebo, for 24 weeks.

Subjects and their physicians were instructed to make all efforts to keep doses of prednisone and other SLE medications constant during the study. Only if medically required, a subject's prednisone dosage was permitted to be increased up to 10 mg/day over baseline dosage for up to 2 weeks. Changes in NSAIDs were allowed, but only if medically required and approved by the treating physician. The protocol for the study was approved by the institutional review board of Triservice General Hospital of Taiwan. All patients gave written informed consent.

Measurements

Serum testosterone, estradiol and cytokine profiles were measured at baseline and at last visit (i.e., after 24 weeks). Different serum cytokines including IL-10, IL-2, IFN-γ, IL-4, and IL-10 in the subjects were measured using ELISA kits (R&D, Minneapolis, Minn.) according to the manufacturer's instructions.

General Statistical Considerations.

Comparisons between groups were done through an independent t-test, while comparisons within groups were done through a paired t-test. All statistical tests were evaluated at a 0.05 level of significance.

Results

Subject Disposition

A total of 32 subjects were randomized in the trial. Of those 32, 17 subjects received 200 mg/day of DHEA, and 15 subjects received a placebo. Fifteen (88%) of the DHEA group and 15 (100%) of the placebo group completed the study.

Baseline Characteristics Demographics

The subject population consisted of Chinese female patients (about 93% premenopausal). The mean age of the subjects was approximately 33 years. Most subjects (about 93%) were using prednisone at the time baseline was determined. Only two subjects had a baseline SLEDAI score of less than 3.

The two treatment groups were well-balanced in terms of age, baseline prednisone dose/use, menopausal status, cytotoxic use and anti-malarial use. See, Table 1. The groups were also well-matched with regard to values at baseline for scoring instruments (data not shown).

TABLE 1
Demographic Summary by Treatment Group.

| | DHEA (n = 17) | | Placebo (n = 15) | | P-value¹ |
|---|---|---|---|---|---|
| Age: | 33.3/32.0 | (10.2) | 32.0/32.0 | (9.6) | 0.672 |
| Mean/Median (SD) | | | | | |
| Age: | 18.0-62.0 | | 18.0-59.0 | | |
| Range | | | | | |
| Prednisone dose (mg/day): | 7.0/7.5 | (3.1) | 6.7/5.0 | (3.1) | 0.732 |
| Mean/Median (SD) | | | | | |
| Prednisone dose (mg/day): | 0.0-10.0 | | 0.0-10.0 | | |
| Range | | | | | |
| Prednisone use at baseline | 14 | (93.3%) | 14 | (93.3%) | 1.000 |
| Pre-menopausal | 14 | (93.3%) | 14 | (93.3%) | 1.000 |
| Cytotoxic use at baseline | 6 | (40.0%) | 6 | (40.7%) | 0.940 |
| Antimalarial use at baseline | 9 | (65.0%) | 10 | (66.6%) | 0.602 |

¹P-values for continuous variables are from an ANOVA comparison, while p-values for categorical variables were done using a chi-square test.

Hormone Determination

As can be seen from Table 2, estradiol levels decreased in both groups (i.e., in those receiving DHEA and those receiving a placebo). The median decrease was slightly larger for the DHEA group than for the placebo group (39.3 vs. 31.5 pg/ml). The difference between treatments in the mean change was considerably greater than for the median. Since the study population was comprised mainly of pre-menopausal women and measurements were not timed in relation to menses, changes in mean and median estradiol levels were probably related to variability in these data and not of clinical relevance.

Mean and median testosterone levels increased in the DHEA group and decreased in the placebo group (46.6 and 39.5 ng/dl compared to −6.6 and −6.5 ng/dl, respectively). See, Table 2. Most subjects in both treatment groups had DHEA-S levels of 0 to 200 mcg/dl at baseline. At the post-baseline visits, approximately 60% of subjects in the DHEA treatment group had levels >1000 mcg/dl. The levels for the remaining 40% were distributed over each of the lower 200 mcg/dl incremental ranges. There was no evidence of elevated DHEA-S levels in the placebo group at baseline or subsequent visits.

TABLE 2
Hormone determination.

| | DHEA | DHEA change | Placebo | Placebo change |
|---|---|---|---|---|
| | baseline | at last visit | baseline | at last visit |
| Estradiol | 123 (104) | −48 (−39) | 116 (103) | −24 (−32) |
| (pg/ml) | | | | |
| Testosterone | 29 (28) | 47 (40) | 30 (28) | −7 (−7)¹ |
| (ng/dl) | | | | |

¹Data presented by mean (median). P-value < 0.05 DHEA vs. placebo. P-value determined through ANOVA comparison.

Cytokine Determination

IL-10 was significantly higher at the time of the last visit in the placebo-treated group as compared to the DHEA-treated group (i.e., placebo group 9.06±7.5 vs. DHEA group 1.89±1.47 pg/ml, p=0.045). See, Table 3. In addition, the reduction in IL-10 from baseline to time of last visit was significant within the DHEA-treated group (i.e., mean baseline concentration of 9.21±6.75 and mean last visit concentration of 1.89±1.47 pg/ml, p=0.029). See, Table 4.

TABLE 3
Between Group Comparison of cytokine profiles.

| | DHEA group | Placebo group | p |
|---|---|---|---|
| IL-1β before | 9.94 ± 8.92 | 7.22 ± 4.24 | 0.498 |
| treatment (pg/ml) | | | |
| IL-10 before | 9.21 ± 6.75 | 8.20 ± 6.25 | 0.577 |
| treatment (pg/ml) | | | |
| IL-1β after | 9.20 ± 6.49 | 9.02 ± 6.83 | 0.942 |
| treatment (pg/ml) | | | |
| IL-10 after | 1.89 ± 1.47 | 9.06 ± 7.50 | 0.045 |
| treatment (pg/ml) | | | |

1. Data was presented by mean ± SD (n = 15 for each group). The p values were analyzed by independent t-test.

TABLE 4
Intra-group comparison of cytokine profiles.

| | DHEA group | p | Placebo group | p |
|---|---|---|---|---|
| IL-1β before | 9.94 ± 8.92 | 0.949 | 7.22 ± 4.24 | 0.441 |
| treatment (pg/ml) | | | | |
| IL-1β after | 9.20 ± 6.49 | | 9.02 ± 6.83 | |
| treatment (pg/ml) | | | | |
| IL-10 before | 9.21 ± 6.75 | 0.029 | 8.20 ± 6.25 | 0.519 |
| treatment (pg/ml) | | | | |
| IL-10 after | 1.89 ± 1.47 | | 9.06 ± 7.50 | |
| treatment (pg/ml) | | | | |

1. Data was presented by mean ± SD (n = 15 for each group). The p values were analyzed by paired t-test.

Discussion

The potential of using DHEA in the treatment of SLE arose from several sets of observations including the female predominance of SLE (see, American College of Rheumatology Ad Hoc Committee, supra), the observation of low circulating levels of DHEA and DHEA-S in patients with active SLE disease (see, Lahita and Jungers, supra), and the possible relation of pregnancy to flare in some patients (see, e.g., Mund, et al., JAMA, 1963, 183:917-20; Petri, et al., Arthritis Rheum. 1991, 34:1538-45; and U.S. Ser. No. 09/710,754 Treatment of SLE with Dehydroepiandrosterone, filed Nov. 10, 2000). Furthermore, DHEA is associated with significantly delayed onset of disease and a reduction in mortality in the NZB/NZW mouse model of SLE. See, Roubinian, et al., Arthritis Rheum., 1979, 22:1162-69, Melez et al., Arthritis Rheum., 1980, 23:41-7, and Lucas et al., J. Clin. Invest., 1985, 75:2091-93. DHEA may have immunomodulatory effects, including a shift from Th2 to Th1, and, thus, a potential benefit in SLE. See, e.g., Daynes, et al., Eur. J. Immunol. 1990, 20:793-802 and Suzuki et al., Clin. Immunol. Immunopathol., 1991, 61:202-11.

Previously, the inventors found significant reductions in the proportion of patients with flare-ups or serious lupus-related adverse events, and improvement in patient global assessments for patients who received DHEA as opposed to those who received placebo. See, Chang, et al., Arthritis Rheum., 2002, 46(11):2924-27.

Multiple mechanisms possibly could be mediating these effects (i.e., on SLE through DHEA treatment) including favorable changes in androgen/estrogen ratio. In addition, Th2/Th1 balance and suppression of inflammatory cytokines could be reasonable mechanisms for DHEA effects. Circulating concentrations of IL-6 are increased in active SLE (see, Linker-Israeli, et al., J. Immunol., 1991, 147:117-23 and Zietz, et al., J. Rheumatol., 2000, 27:911-8), and DHEA has been demonstrated to reduce secretion of IL-6 in vitro from human marrow and mononuclear cells. See, Gordon, et al., Cytokine, 2001, 1695):178-86 and Straub, et al., J. Clin. Endocrinol. Metab., 1998, 83:2012-7. DHEA also suppressed the production of cytokines IL-6 and tumor necrosis factor alpha by Th2 cells which were otherwise stimulated by retrovirus infection. See, Araghi-Niknam, et al., Proc. Soc. Exp. Biol. Med., 1997, 216;386-91. Furthermore, pre-incubation of peripheral blood mononuclear cells (PBMCs) from adult male lupus patients with DHEA reduced the IL-4 production by concanavalin A stimulated PBMCs. See, Tabata, et al., Arch. Dematol. Res., 1997, 287:410-4.

However, in this double-blind study conducted to evaluate the effects of DHEA on cytokine profiles in lupus disease, the inventors found significant reduction of IL-10 in patients' blood after DHEA treatment for 24 weeks.

IL-10 is a B-cell stimulatory cytokine that also inhibits type I cytokine response. See, Fitch, et al., “T-cell mediated immune regulation,” in Fundamental Immunology (Paul, W. E., ed.), Raven, N.Y., pp. 801-835 and Durum, et al., “Proinflammatory cytokines and immunity,” ibid. Several lines of evidence suggest that IL-10 plays a role in the immunopathogenesis of SLE. Lupus patients have increased serum levels of IL-10, which may correlate with disease activity. See, Houssiau, et al., Lupus, 1995, 4:393-395. PBMCs from patients with SLE exhibit increased IL-10 mRNA IL-10 mRNA expression and increased spontaneous IL-10 production. See, Llorente, et al., Arthritis Rheum., 1994, 37:1647-1655 and Richaud-Patin, et al., Rev. Invest. Clin., 1995, 47:267-72. In addition, the peripheral blood of SLE patients contains a significantly increased number of IL-10 secreting cells. See, Hagiwara, et al., Arthritis Rheum., 1996, 39:379-385. Disease activity in SLE patients correlates with the ration of IL-10 secreting cells to IFN-γ secreting cells. Ibid.

The current finding of significant IL-10 suppression by treatment of 200-mg/day dosages of DHEA daily for 24 weeks in adult Chinese female patients with mild to moderate SLE is, thus, thought to explain why DHEA offers meaningful benefit especially to steroid-dependent lupus patients.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

1. A method of modulating a level of IL-10 in a subject, the method comprising:
a) selecting a subject in need of a modulated IL-10 level; and,
b) administering an amount of DHEA to the subject, wherein the amount is effective to modulate the level of IL-10 in the subject.

2. The method of claim 1, the method further comprising:
c) measuring the level of IL-10 in the subject.

3. A method of modulating a level of IL-10 in a subject, the method comprising:
a) administering an amount of DHEA to the subject, wherein the amount is effective to modulate the level of IL-10 in the subject; and,
b) measuring the level of IL-10 in the subject.

4. The method of claim 1, 2, or 3, wherein the modulating comprises decreasing the level of IL-10.

5. The method of claim 4, wherein the level of IL-10 is an elevated level of IL-10.

6. The method of claim 4, wherein the level of IL-10 is a normal level of IL-10.

7. The method of claim 1, 2, or 3, wherein the modulating comprises stabilizing the level of IL-10, wherein the level of IL-10 is an increasing level.

8. The method of claim 1, 2, or 3, wherein the modulating comprises decreasing the rate of increase in the level of IL-10, wherein the level of IL-10 is an increasing level.

9. The method of claim 1, 2, or 3, wherein the level of IL-10 comprises a level of IL-10 in a biological sample of the subject.

10. The method of claim 9, wherein the biological sample comprises a biological fluid.

11. The method of claim 10, wherein the biological fluid comprises one or more of: peripheral blood, serum, plasma, urine, vaginal fluid, semen, saliva, peritoneal fluid, lymphatic fluid, aqueous or vitreous humor, tears, pulmonary effusion or serosal fluid.

12. The method of claim 9, wherein the biological sample comprises a biological tissue.

13. The method of claim 1, 2, or 3, wherein the level of IL-10 comprises a ratio of IL-10 secreting cells to IFN-γ secreting cells in the subject's serum, plasma, or peripheral blood.

14. The method of claim 1, 2, or 3, wherein the subject comprises a human subject.

15. The method of claim 1, 2, or 3, wherein the subject comprises a nonhuman primate.

16. The method of claim 1, 2, or 3, wherein the subject has a medical condition, comprising an increased level of IL-10.

17. The method of claim 16, wherein the condition is systemic lupus erythematosus.

18. The method of claim 16, wherein the condition is rheumatoid arthritis, multiple myeloma, lymphoid malignancy, B-cell lymphoma, Sjögren's syndrome, scleroderma, allergic diseases, HIV/AIDS or plasma cell dyscrasia.

19. The method of claim 1, 2, or 3, wherein the subject has a medical condition, which condition can be ameliorated by decreasing the level of IL-10.

20. The method of claim 1, 2, or 3, wherein the administering comprises oral, intracerebral, intrathecal, intraperitoneal, intramuscular, intravenous, subcutaneous, transdermal, mucosal or ocular administration.

21. The method of claim 20, wherein the administration comprises administering from about 25 mg/day to about 300 mg/day.

22. The method of claim 21, wherein the administration comprises administering from about 100 mg/day to about 250 mg/day.

23. The method of claim 22, wherein the oral administration comprises administering from about 150 mg/day to about 200 mg/day.

24. The method of claim 23, wherein the administration comprises administering about 200 mg/day.

25. The method of claim 20, wherein transdermal administration comprises administration via skin-patch, cream, or topical ointment.

26. The method of claim 1, 2, or 3, wherein administering comprises administration of DHEA and one or more of: a glucocorticoid, a monoclonal antibody specific for IL-10 or a fragment of IL-10, an immunosuppressant, an anti-malarial drug, an alkylating agent, or a chemotherapeutic agent.

27. The method of claim 2 or 3, wherein measuring the level of IL-10 in the subject comprises measuring a basal level of IL-10 in the subject prior to administering the amount of DHEA.

28. The method of claim 27, wherein measuring the level of IL-10 in the subject additionally comprises measuring a level of IL-10 in the subject after administering the amount of DHEA.

29. A method of prophylactically or therapeutically treating one or more medical conditions in a subject, the method comprising modulating a level of IL-10 by:
a) administering a first amount of DHEA to the subject;
b) measuring the level of IL-10 in the subject; and,
c) administering at least a second amount of DHEA to the subject, wherein the second amount is determined based upon the level of IL-10 measured in step (b).

30. The method of claim 29, wherein the medical condition is SLE, rheumatoid arthritis, multiple myeloma, lymphoid malignancy, B-cell lymphoma, Sjögren's syndrome, scleroderma, allergic diseases, HIV/AIDS or plasma cell dyscrasia.
